(19) United States
(12) Patent Application Publication
(10) Pub. No.: US 2026/0144502 A1
(43) Pub. Date: May 28, 2026

(54) CABLE GUIDANCE SYSTEM FOR A COMPUTED TOMOGRAPHY SYSTEM

(71) Applicant: Siemens Healthineers AG, Forchheim (DE)

(72) Inventors: Stefan GROSS, Trabitz (DE); Rita KRUG, Fuerth (DE); Roman ACHLEITNER, Nuernberg (DE); Benjamin LOEWEN, Schwaebisch Gmuend (DE)

(73) Assignee: Siemens Healthineers AG, Forchheim (DE)

(21) Appl. No.: 19/400,645

(22) Filed: Nov. 25, 2025

(30) Foreign Application Priority Data
Nov. 28, 2024 (DE) 10 2024 211 394.8

(51) Int. Cl. A61B 6/03; A61B 6/00; A61B 6/10

(52) U.S. Cl. CPC A61B 6/035; A61B 6/4405; A61B 6/4423; A61B 6/4464; A61B 6/56; A61B 6/102

(57) ABSTRACT

One or more example embodiments relates to a cable guidance system for a computed tomography system including a gantry adjustable in a horizontal plane, wherein a ceiling column, a gantry pivoting arm and a central pivoting arm are configured to guide at least one supply line along a number of longitudinal axes of the at least one supply line, respectively, to a guide connection.

### CROSS-REFERENCE TO RELATED APPLICATION(S)

The present application claims priority under35 U.S.C. § 119 to German Patent Application No. 10 2024 211 394.8, filed Nov. 28, 2024, the entire contents of which is incorporated herein by reference.

### FIELD

One or more example embodiments relates to a cable guidance system for a computed tomography system and a computed tomography system (CT system) for generating tomographic X-ray images.

### RELATED ART

CT systems with movable gantries are being used increasingly in modern medical examination and treatment facilities. The main purpose of the movability of these systems is to allow the typically large scanners (gantries), which take up a lot of space, to be moved. This creates space in the immediate vicinity of the patient for medical staff and/or other equipment or appliances that are used during an examination, treatment and/or intervention. The focus is on the well-being and safety of the patient, but also of the operating personnel and the machines.

In addition, the movability of the gantry also offers the possibility of using it in different treatment rooms, thus reducing investment and maintenance costs in the long term.

The prior art in this field is US 2024/0032 883 A1.

It is known to move gantries along rails so that the gantries can assume predefined positions along the rails. Alternatively, freely movable gantries are also available on the market. While freely movable gantries have a rechargeable on-board power supply, for example in the form of a lithium-ion battery; the challenge with rail-based systems is to provide a wired power supply.

In the case of movable gantries, in particular when they are used in different (typically two) treatment rooms, cable guidance systems must be designed so as to be movable and flexible, so that they can bridge distances of up to 12 m. However, mechanical stress must not impair the service life of the cable guidance system.

In addition, it must be ensured that the cable guidance system cannot cause collisions with the patient, medical staff or surrounding equipment, even when the gantry is being adjusted.

The prior art provides solutions for cable guides that are located in the floor, typically near the rail system. This largely prevents collisions. However, these solutions require specific structural conditions in the hospital environment and are therefore not universally applicable. In addition, they are often not adapted to the hygiene requirements of a medical environment and are expensive.

Alternatively, solutions are known in which supply lines are arranged in a ceiling box (a horizontal guide) using one or more energy supply chains. Here, the rail system runs parallel to the longitudinal axis of the ceiling box. A moving vertical column is provided on the gantry, through which the supply lines are guided downward to the foot of the gantry and connected there. The length of the ceiling box defines the maximum adjustment path for the gantry.

### SUMMARY

One or more example embodiments provides a cable guidance system for a CT system and a computed tomography system for generating tomographic X-ray images with which the above-described disadvantages are avoided. For example, one or more example embodiments provides alternative means for guiding a cable for a movable CT system which enable greater flexibility of movement with a long service life and low construction costs, and increase the freedom of movement of cable-and rail-bound gantries to such an extent that they can be used in different treatment rooms with opposite directions of operation. The creation of a tube cable guide for a movable gantry is one example.

### BRIEF DESCRIPTION OF THE DRAWINGS

Example embodiments will be explained in more detail below with reference to the accompanying figures. Identical components are marked with identical reference characters in the various figures. The figures are generally not to scale. In the drawing:

FIG. 1 shows a rough schematic representation of a CT system.

FIG. 2 shows a displaceable gantry according to the prior art.

FIG. 3 shows a displaceable gantry in accordance with one or more example embodiments.

FIG. 4 shows the cable guidance system in accordance with one or more example embodiments.

FIG. 5 shows a ceiling column of the cable guidance system in accordance with one or more example embodiments.

FIG. 6 shows a route of supply lines in articulated arms in accordance with one or more example embodiments.

FIG. 7 shows the ceiling column with connecting rods in accordance with one or more example embodiments.

### DETAILED DESCRIPTION

A cable guidance system for a computed tomography system in accordance with one or more example embodiments, whose gantry can be adjusted in a horizontal plane, comprises the following components:
a guide connection arranged on the gantry,
a gantry pivoting arm and a central pivoting arm, wherein the gantry pivoting arm is connected to the guide connection via a first joint and to the center articulated arm via a second joint, in each case in a pivotable manner,
a ceiling column with a column head that can be attached to or proximal to a room ceiling and a column body arranged on the column head, wherein the central pivoting arm is attached to a coupling site of the column body facing away from the column head in such a manner as to be able to rotate around the longitudinal axis of the column,
wherein the ceiling column, the gantry pivoting arm and the central pivoting arm are designed so as to guide at least one supply line along their longitudinal axes to the guide connection.

The cable guidance system for a computed tomography system (CT system) therefore comprises multiple components which in cooperation with one another render it possible to guide cables (supply lines) to the gantry of the CT system in a flexible and secure manner. The particular difficulty here is that cables must be guided safely without getting caught on objects or structural features in the room. In particular, repeated bending should also not have a negative effect on the cables. The “cables” are described here as “supply lines” because it is not necessarily only power cables for energy supply and data cables for data transmission that need to be guided, but sometimes also lines for cooling fluids or similar.

The gantry itself does not have to be part of the cable guidance system, nor do the supply lines necessarily have to be part of the cable guidance system. However, the cable guidance system must be attached or secured to the gantry in some way. This site is the guide connection which can be attached or is attached to the gantry. This connection can also be used as a contact point for the supply lines. Basically, two cases are possible in practice. In the first case, supply lines run without connecting sites through the cable guidance system directly into the gantry where they are then connected. In this case, the guide connection is only used for the mechanical connection of the cable guidance system to the gantry. In the second case, a connecting site for the supply lines to the gantry is provided (in particular in the region of the connection). The guide connection can then also be used as a cable connection or generally as a connection of the supply lines.

In general, the guide connection ensures that it is possible to produce a mechanical connection between the cable guidance system and gantry and also that all necessary cables and lines can be guided safely into the gantry in order to ensure a frictionless function of the CT system. By way of example, the guide connection is a rigid or rotatable cable connection, in particular with integrated protection against electromagnetic interference which can be attached or is attached to the gantry.

Gantries frequently have a vertical column for supply lines which run vertically upward from a base of the gantry. The guide connection can be configured so that it can be attached to this column or it can be this column itself.

The cable guidance system comprises between the guide connection and the ceiling column two articulated arms, through which a supply line is safely guided or can be guided. These are the gantry pivoting arm and the central pivoting arm. The names have been selected merely for better differentiation. The articulated arms could also be referred to as the first articulated arm and the second articulated arm. The gantry pivoting arm is located on the gantry, more precisely on the guide connection, and the central pivoting arm is located between the gantry pivoting arm and the ceiling column. The tube cross-section of the articulated arms can be round, square or oval. Articulated arms are known per se in the prior art. They are generally hollow so that supply lines can be guided inside them.

Both articulated arms should be arranged over the gantry so that they cannot collide with the gantry. Since the gantry is not usually part of the cable guidance system, but its dimensions are known (as are the dimensions of the surrounding space), the articulated arms of a cable guidance system for a known gantry in a known space are arranged in such a manner that, when attached to the gantry, they are positioned above it.

The gantry pivoting arm is connected in a pivotable manner both to the guide connection via a first joint and to the central pivoting arm via a second joint (by its ends). This makes it flexible and movable. It is preferred that it can pivot in at least a plane, in particular an essentially horizontal plane, however it is particularly preferred if it can pivot both horizontally and also vertically. The expression “essentially” means here preferably “with a deviation of less than 20°”.

The central pivoting arm is, as mentioned, pivotably connected (at one end) to the gantry pivoting arm via the second joint and (at its other end) to the column body of the ceiling column. It is used as a connecting link between the ceiling column and the gantry pivoting arm, which means that it can guide supply lines from the ceiling column to the gantry pivoting arm. Additional flexibility in guiding the supply lines is rendered possible by the movable arrangement.

The central pivoting arm therefore takes the supply lines from the ceiling column and directs them to the gantry pivoting arm, which in turn directs them to the guide connection. Wherein this guidance through the joints and the movement of the ceiling column compensate for movements of the gantry.

The ceiling column with the column head and column body is a very important element of the cable guidance system. The ceiling column is attached to the room ceiling and is used as a fixed anchor point for the cable guidance system. The column body guides the supply lines from the ceiling to the central pivoting arm. One example would be a robust ceiling column, manufactured from stainless steel, with a hollow column body through which the supply lines can be guided from the column head to the central pivoting arm.

The column head can be attached to or proximal to a room ceiling, which means that it can be fixedly mounted to this ceiling but can also move, for example on a carriage or sled guided on the ceiling. By way of example, the column head can be shaped in such a manner that it can be attached to a running carriage of a horizontal guide on a room ceiling.

The coupling site can be configured in such a manner that it is rotatable relative to the column body. However, it can also be fixed relative to the column body and the entire column body is rotatable relative to the column head. The main thing is that the central pivoting arm is always able to rotate relative to the column head. The coupling site is arranged at a site on the column body facing away from the column head, in other words on the other end of the column body. When a ceiling column is attached to a ceiling in accordance with its intended use, the column head is located at the top of the ceiling column and the coupling site is located at the bottom on the lower end of the column body.

These components consequently support the movability of the gantry of the CT system while simultaneously ensuring that supply lines are guided in a safe and efficient manner. Their flexibility and stability contribute significantly to the reliability and performance of the computed tomography system.

A computed tomography system in accordance with one or more example embodiments is used to generate tomographic X-ray images. It comprises a gantry which is designed so as to be able to be displaced in a horizontal plane, and a cable guidance system in accordance with one or more example embodiments.

Other, particularly advantageous, embodiments and developments of the invention are disclosed in the dependent claims and the description below, wherein the claims of one claim category may also be further developed analogously to the claims and description parts of another claim category, and in particular also individual features of different exemplary embodiments or variants can be combined to form new exemplary embodiments or variants.

In accordance with a preferred cable guidance system, the area of the coupling site is part of the column body. It is preferred that this column body is rotatable relative to the column head around its longitudinal axis, in particular around an angle of rotation greater than 90°. It is preferred that the column body is cylindrical in shape and the central pivoting arm is attached to a peripheral surface of the column body. This part of the peripheral surface forms the coupling site. The central pivoting arm can be fixedly connected to the coupling site so that the central pivoting arm is unable to move relative to the coupling site. However, it can also be arranged so as to be able to move on the coupling site, so that it has a certain amount of play. A fixed arrangement has the advantage that the central pivoting arm is securely held by the ceiling column, preventing it from getting in the way of the travel path of the gantry. An arrangement with play has the advantage that an impact on the central pivoting arm can be better cushioned. It is particularly preferred to attach the central pivoting arm in such a manner that a certain amount of play is possible but which is so small that the central pivoting arm does not hang down in the travel path of the gantry.

In accordance with a preferred cable guidance system, the ceiling column has an energy chain in its column head. This is preferably arranged in such a manner that it allows variable guidance of a supply line around the longitudinal axis of the ceiling column. When a ceiling column is attached in accordance with its intended use, the longitudinal axis runs essentially vertically. The energy chain is then basically located on the side so that the supply line can be guided in a curve on an essentially horizontal plane. The expression “essentially” means here that an incline of a maximum 20° relative to the vertical or horizontal is allowed. It is preferred that the energy chain is fixedly attached to a cable inlet of the column head and can move around a hole in the column body parallel to its longitudinal axis. This is particularly advantageous for the case that the complete column body is mounted in a rotatable manner. The one end of the energy chain is then preferably connected to the column body in order to follow the rotational movement of the column body. The energy chain located in the column head is used to compensate the rotational movement of the column body with the central pivoting arm.

It is preferred that the column head has two interfaces or one universal interface in order to be able to be fixedly mounted on the ceiling or a horizontal adjustment option.

A preferred cable guidance system is characterized by virtue of the fact that the column head is wider than the column body, preferably in a spatial orientation at least twice as wide, and has a space for receiving the energy chain, in which the energy chain can move between its extreme positions. It is to be noted at this point that an energy chain requires a certain amount of free space during its movement. In this case, the free space should be sufficiently large so that the energy chain can move freely between the extreme positions of the rotation of the coupling site (or of the column body). The column head should therefore have in its interior a free space which corresponds to the space covered by the energy chain between the extreme positions of the rotation of the coupling site (or of the column body). Since the column body does not need to be larger than necessary for its storage and for guiding supply lines and a required stability, the column head with its free space inside should generally be larger.

A preferred cable guidance system is characterized by virtue of the fact that the gantry pivoting arm and the central pivoting arm run at least in part in a plane. This plane is preferably essentially orthogonal to the longitudinal axis of the ceiling column. The expression “essentially” means here that a deviation of a maximum 10° relative to the angle 90° is allowed.

In accordance with a preferred cable guidance system, the ceiling column is height-adjustable. It is preferred that it has the connecting rods between the column head and the coupling site. These connecting rods are arranged in particular in a circular manner around the longitudinal axis of the ceiling column. The connecting rods can form a closed circle or merely a framework. They are preferably surrounded by a wall. The construction can be adapted to the height of the room by cutting the connecting rods to length.

In accordance with a preferred cable guidance system, the second joint is designed in such a manner that it renders possible positions in which the gantry pivoting arm and the central pivoting arm enclose an angle between 10° and 170° and/or wherein the first joint is designed in such a manner that it renders possible positions in which the gantry pivoting arm and the gantry enclose an angle between 10° and 160°. As a result, the articulated arms should have sufficient movability for most applications.

A preferred cable guidance system is characterized by virtue of the fact that the first joint and/or the second joint are surrounded by a flexible wall which is preferably formed via corrugated tubes. This wall both prevents damage to the supply lines and also renders it possible to clean or sterilize the cable guidance system in a simple manner.

A preferred cable guidance system is characterized by virtue of the fact that the length of the gantry pivoting arm corresponds 65% to 75% to the length of the central pivoting arm, and/or wherein the gantry pivoting arm is a maximum 1600 mm and the central pivoting arm a maximum 2300 long. The arm lengths can vary in length depending upon the spatial situation in order to adapt the functionality according to the space.

A preferred cable guidance system is characterized by virtue of the fact that, in the case of the predetermined dimensions for the gantry and a predetermined point for the guide connection on the gantry, the gantry pivoting arm and guide connection are dimensioned and configured in such a manner that when the guide connection is attached in accordance with its intended use to the gantry the first joint lies essentially over a central axis of rotation of the gantry. The first joint should therefore be positioned centrally over the gantry. This is advantageous in the rotated situation of the gantry.

A preferred cable guidance system comprises a ceiling-mounted horizontal guide which runs above the gantry and extends in its longitudinal axis parallel to the movement direction of the gantry. Such a horizontal guide is known in the prior art. It is preferred that a running carriage which can be adjusted in the longitudinal direction of the horizontal guide and to which the column head is attached is arranged on the horizontal guide. Alternatively, the column head can also be configured as a running carriage. This embodiment is particularly advantageous if the gantry must cover comparatively long paths. By way of example, in the case of a two-room situation, supply lines can, as a result, be organized in such a manner that after the gantry has passed through a door, in other words in the case of a room change, the doors can be completely closed again.

A preferred cable guidance system is characterized by virtue of the fact that the horizontal guide comprises an energy chain for guiding a number of supply lines. Such horizontal guides are known in the prior art.

A preferred computed tomography system comprises a rail system on which the gantry is arranged in a displaceable manner. Such rail systems are known in the prior art.

In accordance with a preferred computed tomography system, the gantry is designed so as, during an adjusting movement along the rail system, to perform a rotation of 180° around a vertical axis running through the iso-center of the gantry.

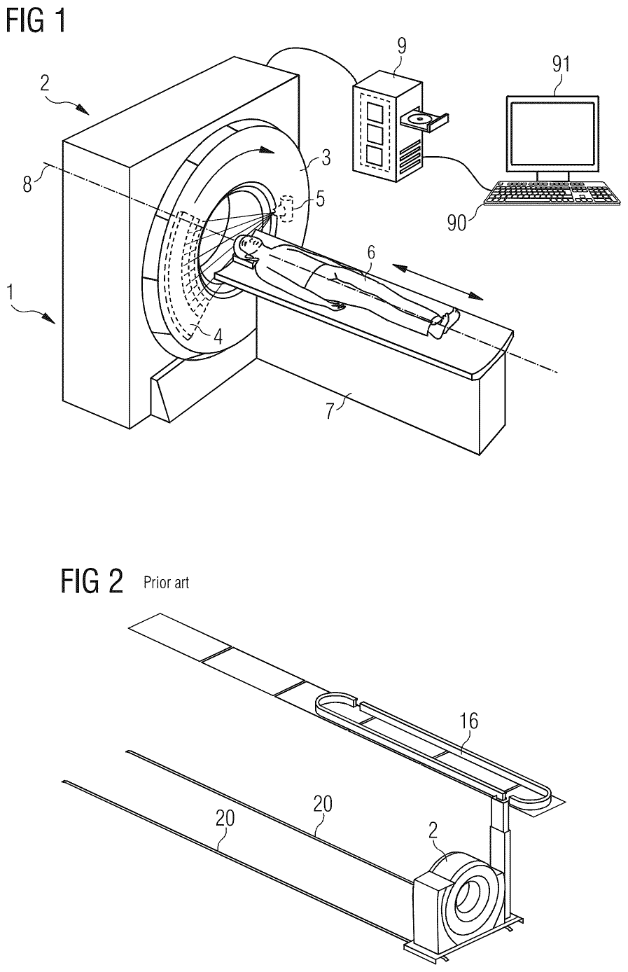

FIG. 1 shows a computed tomography system (CT system 1) with a radiation detector 4 and an X-ray source 5. The X-ray source 5 is designed so as to expose the radiation detector 4 to X-ray radiation. The illustrated CT system 1 comprises a gantry 2 with a rotor 3. The rotor 3 comprises the X-ray source 5 and the radiation detector 4 which is designed so as to detect X-ray radiation.

The rotor 3 is rotatable around the axis of rotation 8. A patient 6 is positioned on the patient couch 7 and can be moved along the axis of rotation 8 through the gantry 2. The computing unit 9 is provided in order to control the CT system 1 and/or to generate sets of image data based on signals detected by the radiation detector 4.

A set of (raw) X-ray image data of the patient 6 is usually recorded from a number of angular directions via the radiation detector 4. Subsequently, based on the set of (raw) X-ray image data, it is possible via a mathematical method, by way of example comprising a filtered back projection or an iterative reconstruction method, to reconstruct a set of image data.

The computing unit 9 is used here as a control facility 9 for controlling the CT system 1. An input facility 90 and an output facility 91 are connected to this computing unit 9. The input facility 90 and the output facility 91 can, for example, enable interaction by a user or the display of a set of generated image data.

FIG. 2 shows a displaceable gantry 2 according to the prior art. The gantry 2 is positioned in a displaceable manner on a rail system 20 with two parallel rails. Cables are guided via a horizontal guide 16 on the ceiling to the gantry 2, so that the gantry can be supplied with energy and send data in every possible position on the rail system 20.

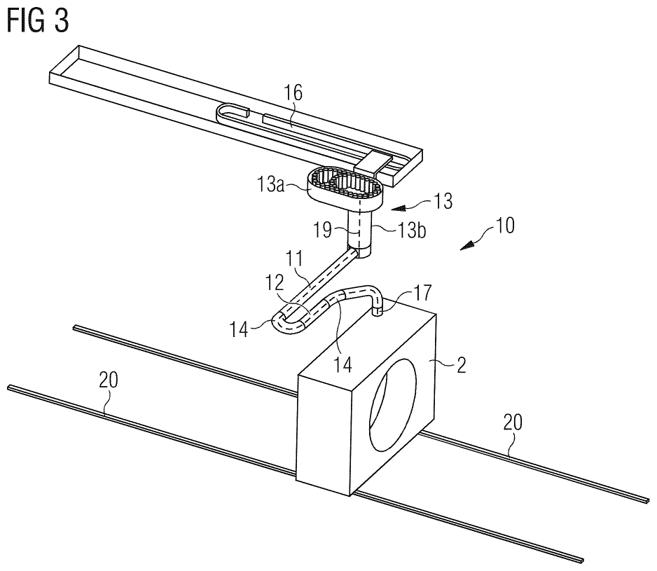

FIG. 3 illustrates a displaceable gantry 2 in accordance with one or more example embodiments, which is mounted in a displaceable manner on a rail system 20 with two parallel rails. A supply line 19 is guided initially via a horizontal guide 16 on the ceiling, and then guided through the cable guidance system 10 to the gantry. In the following figures, this cable guidance system 10 is represented somewhat greater and in more detail.

The cable guidance system 10 comprises a guide connection 17 arranged on the gantry 2, a gantry pivoting arm 12 and a central pivoting arm 11 and a ceiling column 13. The gantry pivoting arm 12 is connected to the guide connection 17 via a first joint 14 and to the central pivoting arm 11 via a second joint 14, in each case in a pivotable manner. The ceiling column 13 is attached by its column head 13a to the horizontal guide 16. The column body 13b, to which the central pivoting arm 11 is attached, is located on the column head 13a and in fact at a coupling site 13c of the column body 13b facing away from the column head 13a. The central pivoting arm 11 is attached in such a manner as to be able to rotate around the longitudinal axis L of the column. The ceiling column 13, the gantry pivoting arm 12 and the central pivoting arm 11 are designed so as to guide the supply line 19 along its longitudinal axis to the guide connection 17, as indicated by the dashed line.

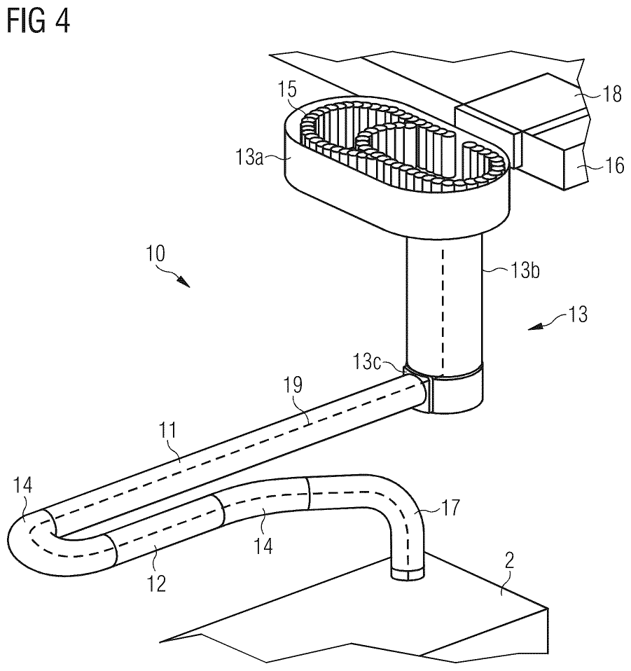

FIG. 4 illustrates the cable guidance system 10 of FIG. 3 in accordance with one or more example embodiments. In this case, the energy chain 15 is easily recognizable in the column head 13a and is represented here in its two extreme positions, in other words in the positions which it assumes if the column body 13b is rotated a maximum to the left and to the right relative to the column head 13a.

FIG. 5 shows the ceiling column 13 of the cable guidance system 10 according to FIGS. 3 and 4 even larger. In this case, the longitudinal axis L is indicated, around which the central pivoting arm 11 rotates (double arrow), the central pivoting arm being attached at the coupling site 13c to the column body 13b. As already mentioned above, in this example, the entire column body 13b rotates around the column head 13a with the central pivoting arm 11 fixedly mounted on it. In this representation, it is also easily recognizable how the column head 13a is displaceably attached to the running carriage 18 of the horizontal guide 16.

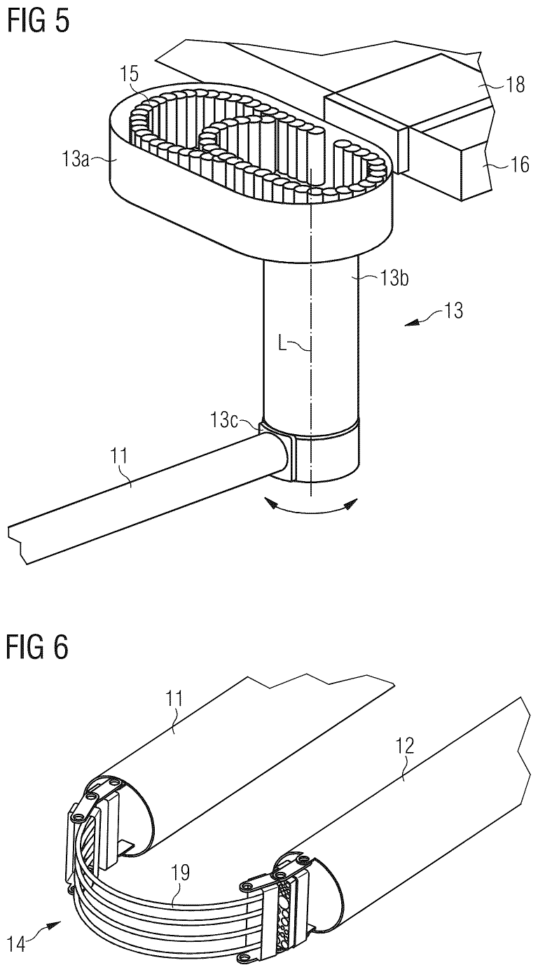

FIG. 6 shows the route of the supply lines 19 in the articulated arms 11, 12. The guide of the supply lines 19 in the joint 14 between the central pivoting arm 11 and the gantry pivoting arm 12 is also supported here by an energy chain, of which only a few links are illustrated.

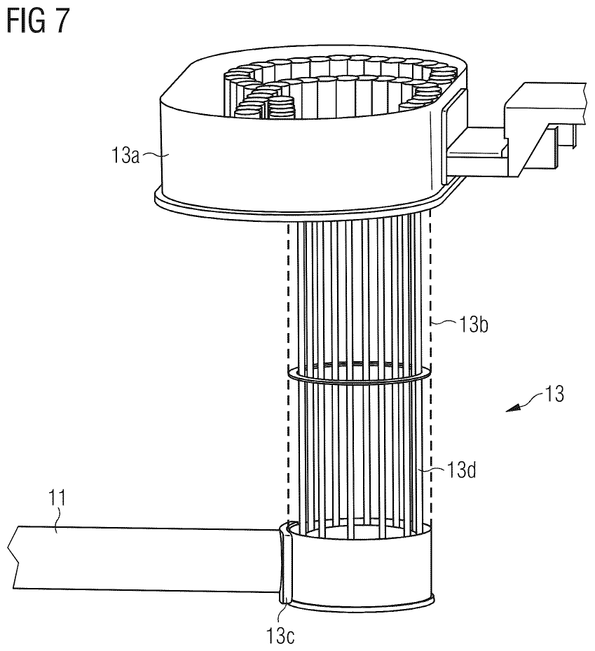

FIG. 7 shows the ceiling column 13 with connecting rods 13d which form the supporting structure of the column body 13b. They can be cut to length to match the dimensioning of the column body 13b and should be surrounded by a wall, so that the column body 13b can be easily disinfected.

Finally, it should be noted once again that the invention described in detail above are merely exemplary embodiments which can be modified in various ways by the person skilled in the art without departing from the scope of the invention. Furthermore, the use of the indefinite article “a” or “an” does not exclude that the relevant features may also be present multiple times. Likewise, terms such as “unit” do not exclude that the relevant components can comprise multiple interacting sub-components, which can also be distributed spatially where appropriate. The term “a number” is to be read as “at least one”. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

When a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc).

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A cable guidance system for a computed tomography system including a gantry adjustable in a horizontal plane, the cable guidance system comprising:
a guide connection on the gantry;
a gantry pivoting arm and a central pivoting arm, the gantry pivoting arm being pivotally connected to the guide connection via a first joint and pivotally connected to the central pivoting arm via a second joint; and
a ceiling column including a column head and a column body, the column head being attachable or proximal to a room ceiling, the column body being on the column head, the central pivoting arm being attached to a coupling site of the column body and rotatable around a longitudinal axis of the ceiling column, the coupling site facing away from the column head,
wherein the ceiling column, the gantry pivoting arm and the central pivoting arm are configured to guide at least one supply line along a number of longitudinal axes of the at least one supply line, respectively, to the guide connection.

2. The cable guidance system of claim 1, wherein the column body is rotatable relative to the column head around a longitudinal axis of the column body.

3. The cable guidance system of claim 1, wherein the ceiling column includes,
an energy chain in the column head, the energy chain configured to allow variable guidance of a supply line around the longitudinal axis of the ceiling column.

4. The cable guidance system of claim 3, wherein the column head is wider than the column body and includes a space to receive the energy chain.

5. The cable guidance system of claim 1, wherein the gantry pivoting arm and the central pivoting arm extend at least in part in a plane.

6. The cable guidance system of claim 1, wherein the ceiling column is height-adjustable.

7. The cable guidance system of claim 1, wherein at least one of
the second joint is configured to permit the gantry pivoting arm and the central pivoting arm to enclose an angle between 10° and 170°, or the first joint is configured to permit the gantry pivoting arm and the gantry to enclose an angle between 10° and 160°.

8. The cable guidance system of claim 1, wherein at least one of the first joint or the second joint is surrounded by a flexible wall.

9. The cable guidance system of claim 1, wherein at least one of
a length of the gantry pivoting arm corresponds to 65% to 75% of a length of the central pivoting arm, or the length of the gantry pivoting arm is at most 1600 mm and the length of the central pivoting arm is at most 2300 mm.

10. The cable guidance system of claim 1, wherein the gantry pivoting arm and the guide connection are dimensioned and configured such that, when the guide connection is attached to the gantry, the first joint is essentially over a central axis of rotation of the gantry.

11. The cable guidance system of claim 1, further comprising:
a ceiling-mounted horizontal guide extending above the gantry and along a longitudinal axis of the ceiling-mounted horizontal guide, the longitudinal axis of the ceiling-mounted horizontal guide being parallel to a movement direction of the gantry.

12. The cable guidance system of claim 11, wherein the horizontal guide comprises an energy chain to guide a number of supply lines.

13. A computed tomography system for generating tomographic X-ray images, the computed tomography system comprising:
a gantry displaceable in a horizontal plane; and
the cable guidance system of claim 1.

14. The computed tomography system of claim 13, further comprising:
a rail system, the gantry being on the rail system.

15. The computed tomography system of claim 14, wherein the gantry is configured to perform a rotation of 180° around a vertical axis running through an iso-center of the gantry during an adjusting movement along the rail system.

16. The cable guidance system of claim 2, wherein the column body is rotatable around an angle of rotation greater than 90°.

17. The cable guidance system of claim 2, wherein the column body is cylindrical and the central pivoting arm is fixedly attached to a peripheral surface of the column body forming the coupling site.

18. The cable guidance system of claim 3, wherein the energy chain is fixedly attached to a cable inlet of the cable head and is moveable around a hole in the column body.

19. The cable guidance system of claim 5, wherein the gantry pivoting arm and the central pivoting arm extend in a plane essentially orthogonal to the longitudinal axis of the ceiling column.

20. The cable guidance system of claim 8, wherein the flexible wall is formed via corrugated tubes.
